(19) United States
(12) Patent Application Publication
(10) Pub. No.: US 2023/0355268 A1
(43) Pub. Date: Nov. 9, 2023

(54) IMAGING APPARATUS

(71) Applicant: TERUMO KABUSHIKI KAISHA, Tokyo (JP)

(72) Inventors: Takito INUKAI, Isehara Kanagawa (JP); Yuji ONIMURA, Fujinomiya Shizuoka (JP)

(21) Appl. No.: 18/352,282

(22) Filed: Jul. 14, 2023

Related U.S. Application Data
(63) Continuation of application No. PCT/JP2021/045049, filed on Dec. 8, 2021.

(30) Foreign Application Priority Data
Jan. 18, 2021 (JP) 2021-005616

(51) Int. Cl. A61B 17/34; A61B 8/00; A61B 8/08

(52) U.S. Cl. CPC A61B 17/3403; A61B 8/403; A61B 8/085; A61B 8/54; A61B 2017/3405

(57) ABSTRACT

An imaging apparatus includes a base portion having a space in which a part of a human body is placed, a probe body attached to the base portion and including an imaging unit that faces the space and is configured to acquire a cross-sectional image of the part of the human body when the part of the human body is placed in the space, a first pressing portion attached to the base portion and by which the part of the human body can be pressed against the imaging unit, and a controller configured to control a pressure applied to the part of the human body by the first pressing portion.

### CROSS-REFERENCE TO RELATED APPLICATION

This application is a continuation of International Patent Application No. PCT/JP2021/045049 filed Dec. 8, 2021, which is based upon and claims the benefit of priority from Japanese Patent Application No. 2021-005616, filed on Jan. 18, 2021, the entire contents of which are incorporated herein by reference.

### FIELD

The present disclosure relates generally to an imaging apparatus and an automatic puncture apparatus.

### BACKGROUND

In order to secure an access site for drug administration and endovascular treatment, vascular puncture for puncturing a human body with an injection needle is performed. In the vascular puncture, an operator cannot visually observe a blood vessel from a skin surface, and thus guesses a position of the blood vessel using standard knowledge of blood vessel locations and skill such as tactile perception of blood vessel pulsation. However, a vascular puncture failure often occurs, which causes physical and mental distress to a patient.

In order to specify a puncture location on a skin surface, a technique for visualizing a location of a blood vessel, such as near-infrared imaging, ultrasound echo, photoacoustic imaging, or the like, is used. For example, an ultrasound device can generate a cross-sectional image of a human body. An operator alternately views a monitor showing the generated cross-sectional image and a puncture location while pressing the ultrasonic probe against the arm of the patient using one hand and performing puncture with a puncture needle using the other hand. For this reason, the operator requires a high skill. To assist in the puncture operation, there is a known ultrasound echographic apparatus provided with a belt for attaching an ultrasonic probe to a human body.

In order to acquire a fine cross-sectional image using an ultrasound device, it is necessary to bring the imaging unit included in the ultrasonic probe into close contact with a human body so that there is no air gap between the imaging unit and the skin surface. However, there is a possibility that the imaging unit cannot be sufficiently brought into close contact with the human body even if the ultrasonic probe is attached to the human body with a belt or the like.

### SUMMARY OF THE INVENTION

Embodiments of this disclosure provide an imaging apparatus capable of reliably bringing an imaging unit of an ultrasonic probe into close contact with a human body.

An imaging apparatus in one embodiment includes a base portion having a space in which a part of a human body is placed, a probe body attached to the base portion and including an imaging unit that faces the space and is configured to acquire a cross-sectional image of the part of the human body when the part of the human body is placed in the space, a first pressing portion attached to the base portion and by which the part of the human body can be pressed against the imaging unit, and a controller configured to control a pressure applied to the part of the human body by the first pressing portion.

In the imaging apparatus configured as described above, the human body inserted into the base portion can be pressed against the imaging surface by the pressing portion, so that a clear cross-sectional image can be acquired by the imaging unit being reliably brought into close contact with the human body.

### BRIEF DESCRIPTION OF THE DRAWINGS

FIG. 1 is a front view of an imaging apparatus according to an embodiment.

FIG. 2 is a cross-sectional view of a base portion having a probe body.

FIG. 3 is a cross-sectional view of the base portion in a state where a pressing portion is inflated.

FIG. 4 is a view illustrating an imaging surface of the probe body and a positional relationship with an arm for which a cross-sectional image is to be acquired.

FIG. 5 is a side view of the base portion.

FIG. 6 is a side view of the base portion in a state where a first base member and a second base member are mutually opened.

FIG. 7 is a cross-sectional view of the base portion in a state where the arm is inserted and the pressing portion is inflated.

FIG. 8 is a hardware block diagram of the imaging apparatus.

FIG. 9 is a flowchart of processing performed by the imaging apparatus.

FIG. 10 is a view illustrating a positional relationship between a position of center of gravity of a blood vessel and an imaging position.

FIG. 11 is a cross-sectional view of a base portion of an imaging apparatus according to a first modification.

FIG. 12 is a cross-sectional view of a base portion in a state where an arm is inserted into the imaging apparatus according to the first modification and a pressing portion is inflated.

FIG. 13 is a side view of a state in which the base portion of the imaging apparatus according to the first modification is opened.

FIG. 14 is a side view of a base portion of an imaging apparatus according to a second modification.

FIG. 15 is a schematic view of an automatic puncture apparatus.

### DETAILED DESCRIPTION

Hereinafter, embodiments of the present disclosure will be described with reference to the accompanying drawings. In some cases, dimensional ratios in the drawings may be exaggerated and different from actual ratios for convenience of description.

An imaging apparatus 10 according to an embodiment is used when an arm of a human body is punctured, and is capable of acquiring and displaying a cross-sectional image of the arm.

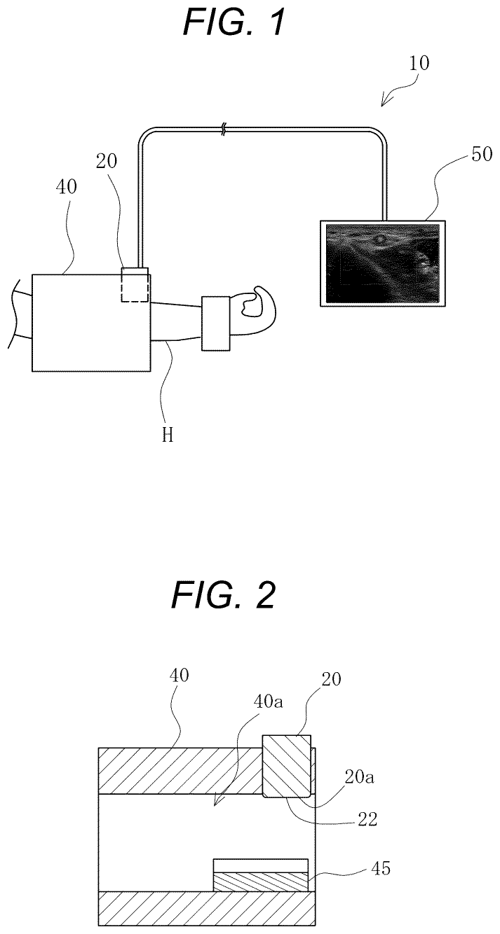

As illustrated in FIGS. 1 and 2, the imaging apparatus 10 includes a base portion 40 that holds an arm H, and a display unit 50 that displays the acquired cross-sectional image. The base portion 40 includes a probe body 20 having an imaging unit 22 that comes into contact with the skin surface to acquire the cross-sectional image of the arm H.

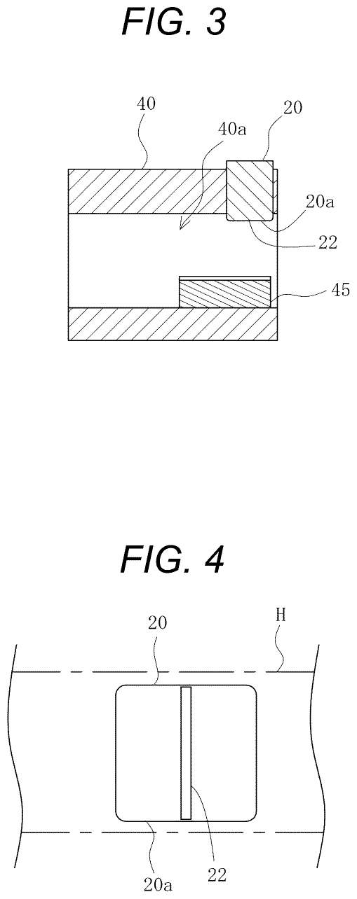

The base portion 40 is formed in a tubular shape and has an internal space 40a penetrating the base portion 40. The probe body 20 has an imaging surface 20a along which the imaging unit 22 is disposed and facing the internal space 40a. A pressing portion 45 is provided on an inner peripheral surface of the base portion 40 to face the imaging surface 20a. The pressing portion 45 is formed with a bag-shaped cuff that inflates by air supply and can inflate so that the inner peripheral surface 45a approaches the imaging surface 20a as illustrated in FIG. 3.

As illustrated in FIG. 4, the imaging unit 22 extends along one direction at a central portion of the imaging surface 20a of the probe body 20 and extends over substantially the entire width thereof. The imaging unit 22 is an echographic apparatus that includes a transducer that generates an ultrasound wave and obtains a cross-sectional image of the inside of the human body by detecting the reflected wave. In an embodiment, the cross-sectional image orthogonal to the axial direction of a blood vessel is acquired, and thus, the imaging unit 22 is disposed such that the length direction thereof is orthogonal to the length direction of the arm H.

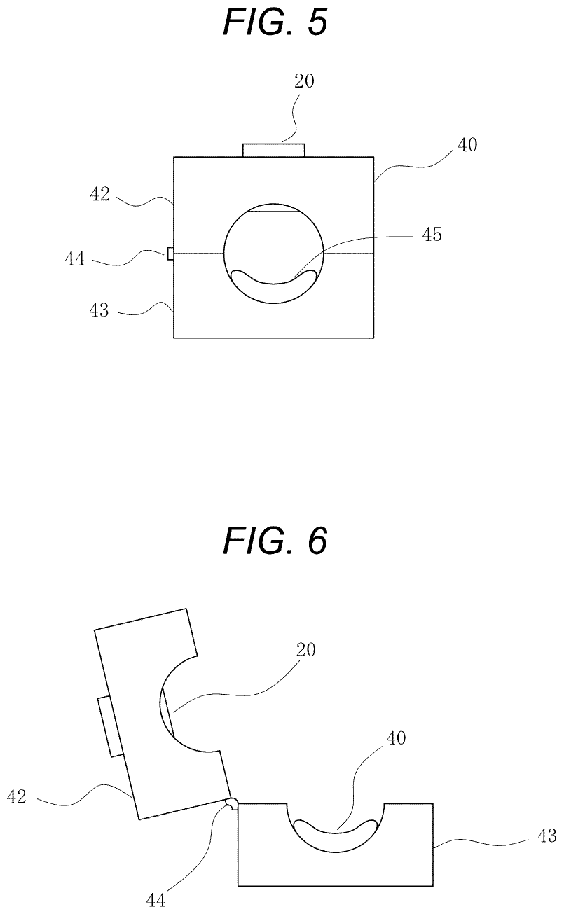

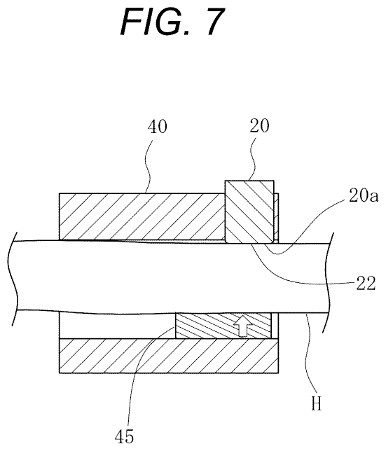

As illustrated in FIG. 5, the base portion 40 is formed by connecting a first base member 42 and a second base member 43 by a hinge portion 44. The inner surface of the internal space 40a of the base portion 40 is divided into two in the circumferential direction by the first base member 42 and the second base member 43. As illustrated in FIG. 6, the first base member 42 and the second base member 43 can be opened and closed around the hinge portion 44. As a result, the internal space 40a can be opened, and the arm H can be easily taken in and out. Further, the base portion 40 can be opened, and thus, a portion facing the internal space 40a of the base portion 40, such as the imaging surface 20a and the pressing portion 45, can be easily cleaned and sterilized.

By inflating the pressing portion 45 in a state where the arm H is inserted into the internal space 40a of the base portion 40, the inner peripheral surface 45a of the pressing portion 45 moves toward the imaging surface 20a, and a pressure toward the imaging surface 20a is applied to the arm H. Thus, the arm H is pressed against and in close contact with the imaging surface 20a. This eliminates an air gap between the imaging unit 22 and the arm H and enables the imaging unit 22 to reliably acquire the cross-sectional image of the arm H.

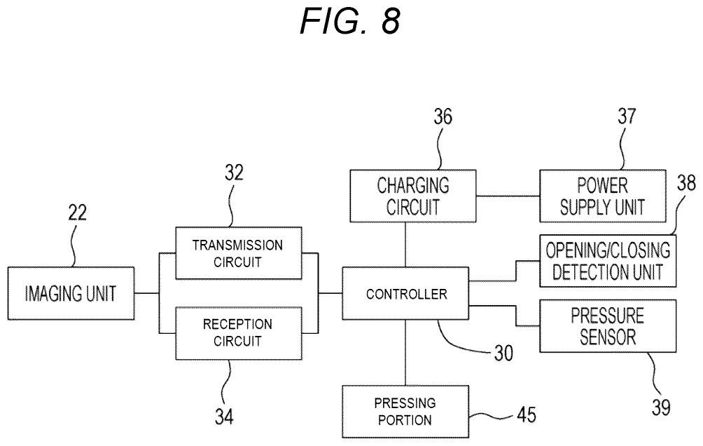

How much the pressing portion 45 is inflated is controlled according to a state of the cross-sectional image to be acquired. As illustrated in FIG. 8, the imaging apparatus 10 includes the imaging unit 22 that comes into contact with a skin surface to acquire a cross-sectional image of a human body, the controller 30 that controls the imaging unit 22 and the pressing portion 45, and the pressing portion 45 connected to the controller 30. The controller 30 is connected to the imaging unit 22 via a transmission circuit 32 and a reception circuit 34 and can cause the imaging unit 22 to acquire a cross-sectional image and receive the acquired cross-sectional image. The controller 30 is connected to a power supply unit 37 including a rechargeable battery via a charging circuit 36. The controller 30 is connected to an opening/closing detection unit 38 that detects an opened/closed state of the first base member 42 and the second base member 43 of the base portion 40, and a pressure sensor 39 that detects a pressure applied to the pressing portion 45. For example, the opening/closing detection unit 38 is a switch or the like, that is switched on and off as the first base member 42 and the second base member 43 come into and out of contact with each other.

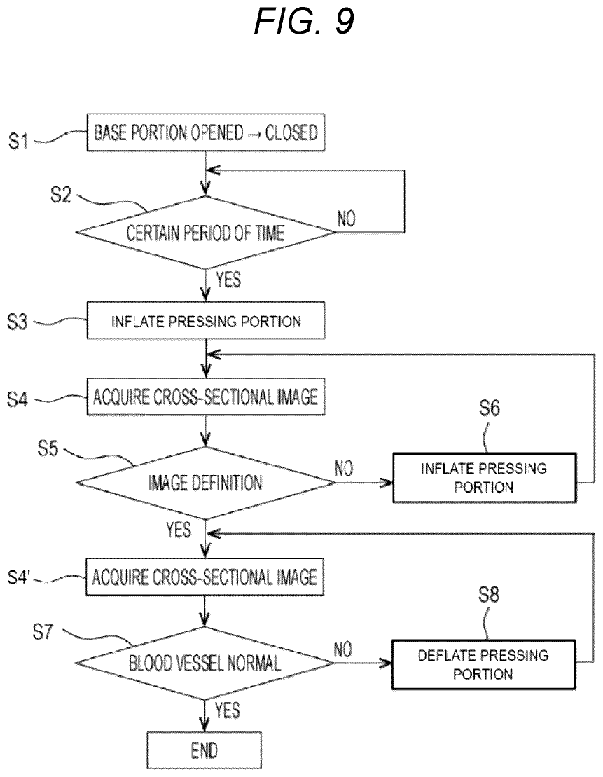

A process for adjusting the inflated state of the pressing portion 45 will be described. As illustrated in FIG. 9, if the opening/closing detection unit 38 detects that the first base member 42 and the second base member 43 of the base portion 40 are changed from a mutually opened state to a closed state (S1), the controller 30 inflates the pressing portion 45 (S3) after a lapse of a certain period of time (S2). The controller 30 inflates the pressing portion 45 until a pressure detected by the pressure sensor 39 reaches a predetermined value. As a result, the internal space 40a of the base portion 40 is reduced, and the arm H is pressed against the imaging surface 20a. In this state, the controller 30 causes the imaging unit 22 to acquire a cross-sectional image (S4).

The controller 30 performs image analysis on the cross-sectional image acquired by the imaging unit 22 to determine the quality the image, e.g., clarity, sharpness, or the like (S5). The quality of an image can be determined by any method. For example, the controller 30 determines the sharpness of an image by checking its brightness distribution. More specifically, the controller 30 extracts multiple lines from an image along one direction, and determines that the quality of the image (i.e., sharpness) is sufficient if the number of lines with a change in the brightness distribution is above a predetermined level. In a case where the sharpness of the image is lower than a certain level, that is, in a case where the image is unclear, it is assumed that pressing of the arm H against the imaging surface 20a by the pressing portion 45 is weak, and the controller 30 further inflates the pressing portion 45 (S6). As a result, the internal space 40a of the base portion 40 becomes smaller, and the arm H is more strongly pressed against the imaging surface 20a, so that a clearer cross-sectional image can be easily obtained. When the pressing portion 45 is further inflated, the controller 30 causes the imaging unit 22 to acquire the cross-sectional image again (S4).

Furthermore, the controller 30 detects a cross-sectional shape of the blood vessel from the image by analyzing the cross-sectional image acquired by the imaging unit 22. The controller 30 detects a region of the blood vessel in the image and sets the shape of the region as the shape of the blood vessel. In order to detect the region recognized as the blood vessel in the image, it is possible to prepare a large number of images of the same type and use a machine learning or deep planning method. In addition, it is also possible to detect a region with blood flow using the Doppler method in the imaging unit 22 and recognize the region as the region of the blood vessel.

When the region of the blood vessel is detected from the cross-sectional image, it is necessary to distinguish an artery from a vein. An artery can be distinguished from a vein on the basis of a position of the bone of the arm H appearing in the cross-sectional image. In addition, in a case where the region with the blood flow is detected by the Doppler method, an artery can be distinguished from a vein by a direction of the blood flow.

After Step S5, the controller 30 causes the imaging unit 22 to acquire another cross-sectional image (S4′), and then determines whether the cross-sectional shape of the blood vessel is nearly circular and normal (S7). In an embodiment, to puncture a vein, the controller 30 determines the cross-sectional shape of the blood vessel recognized as a vein. In order to detect a blood vessel deformed in the acquired cross-sectional image, the controller 30 determines whether there is a difference between the previously acquired and recorded cross-sectional image and the next acquired cross-sectional image. The controller 30 can determine the deformation on the basis of a predetermined threshold or on the basis of machine learning. Furthermore, the controller 30 may confirm whether the deformation has been resolved by a threshold or machine learning by comparing the cross-sectional image acquired again with the cross-sectional image acquired first. Here, in a case where the cross-sectional shape of the blood vessel is not circular but has a deformed shape, it is assumed that pressing of the arm H by the pressing portion 45 is too strong, and the controller 30 deflates the pressing portion 45 (S8). As a result, a pressing force of the arm H by the pressing portion 45 is weakened, which resolves a state in which the blood vessel is deformed. After deflating the pressing portion 45, the controller 30 causes the imaging unit 22 to acquire the cross-sectional image again (S4′).

The controller 30 may determine whether the position of the blood vessel is moved from the cross-sectional image acquired by the imaging unit 22 by movement of the imaging unit 22 and the pressing portion 45. In order to detect the movement of the blood vessel from the acquired cross-sectional image, the controller 30 detects whether there is a difference between the previously acquired and recorded cross-sectional image and the next acquired cross-sectional image. The controller 30 can determine the movement on the basis of a predetermined threshold or on the basis of machine learning. Furthermore, the controller 30 may check whether the movement of the blood vessel has been eliminated by a threshold or machine learning by comparing the cross-sectional image acquired again with the cross-sectional image acquired first. Here, in a case where the movement of the blood vessel is detected, the controller 30 operates the pressing portion 45 so as to make the internal space 40a of the base portion 40 larger in the moving direction of the blood vessel or in the direction opposite to the moving direction of the blood vessel by inflating or deflating part of the pressing portion 45 in a circumferential direction. In this event, part of the pressing portion 45 on the opposite side in the circumferential direction may be deflated with the inflation of part of the pressing portion 45 in the circumferential direction, or part of the pressing portion 45 on the opposite side in the circumferential direction may be inflated with the deflation of part of the pressing portion 45 in the circumferential direction.

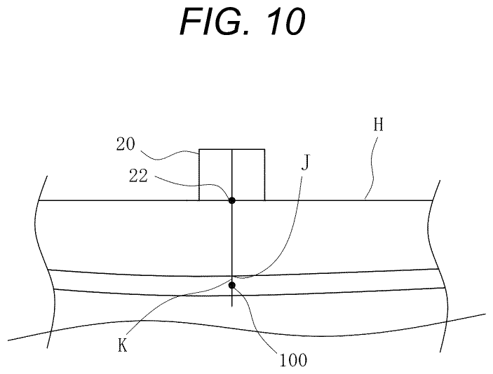

The position of the blood vessel can be specified by detecting a position of the center of gravity of the blood vessel from the cross-sectional image. The controller 30 detects the position of the blood vessel in the image by analyzing the acquired cross-sectional image. The controller 30 detects the region of the blood vessel in the image and sets the position of its center of gravity 100 as the position of the blood vessel as illustrated in FIG. 10. In order to detect the region of the blood vessel in the image, it is possible to prepare a large number of images of the same type and use a machine learning or deep planning method. In addition, it is also possible to detect a region with blood flow by the Doppler method in the imaging unit 22 and recognize the region as the region of the blood vessel. When the region of the blood vessel is detected from the cross-sectional image, it is necessary to distinguish an artery from a vein. An artery can be distinguished from a vein on the basis of a position of the bone of the arm H appearing in the cross-sectional image. In addition, in a case where the region with the blood flow is detected by the Doppler method, an artery can be distinguished from a vein by a direction of the blood flow. Furthermore, the position of the blood vessel is not limited to the position of the center of gravity and may be based on an inner surface position J (see FIG. 10) of the blood vessel located between the blood vessel to be punctured and the imaging unit 22 or a position K in a membrane of the blood vessel.

In this manner, by adjusting the force with which the pressing portion 45 presses the arm H against the imaging surface 20a by the controller 30, it is possible to acquire a favorable cross-sectional image and perform reliable puncture assistance. Note that the flow of adjusting the pressing portion 45 by the controller 30 is not necessarily required, and for example, the pressing portion 45 may be manually adjusted.

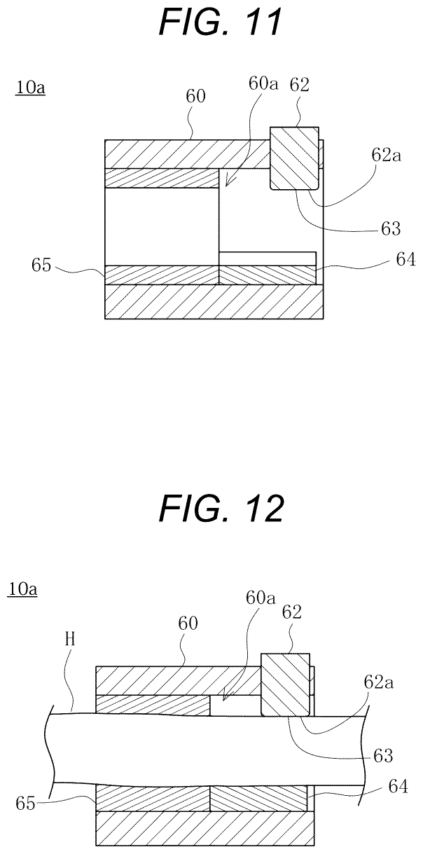

Next, an imaging apparatus 10a of a first modification will be described. As illustrated in FIG. 11, a probe body 62 is provided on a base portion 60 of the imaging apparatus 10a according to the present modification. The probe body 62 is disposed such that an imaging surface 62a along which an imaging unit 63 is disposed faces an internal space 60a. The probe body 62 is disposed near one end in a penetrating direction of the internal space 60a. The base portion 60 has a pressing portion 64 at a position facing the imaging surface 62a. In addition, the base portion 60 has an entire circumference pressing portion 65 over the entire circumference of the inner circumference on the other end side opposite to one end of the internal space 60a in which the probe body 62 is disposed. The pressing portion 64 and the entire circumference pressing portion 65 can inflate toward the inner circumference side.

As illustrated in FIG. 12, by inflating the pressing portion 64 and the entire circumference pressing portion 65 in a state where the arm H is inserted into the base portion 60, an inner circumferential surface 64a of the pressing portion 64 and an inner circumferential surface 65a of the entire circumference pressing portion 65 are in close contact with the arm H. This results in pressing the arm H against the imaging surface 62a while pressing the arm H so as not to move, so that the cross-sectional image of the arm H can be reliably acquired by the imaging unit 63. The entire circumference pressing portion 65 in close contact with the entire circumference of the arm H is disposed closer to the shoulder side of the arm H than the imaging surface 62a. A position closer to the wrist side of the arm H than the imaging surface 60a is punctured, so that it is possible to prevent the entire circumference pressing portion 65 from interfering with the puncture.

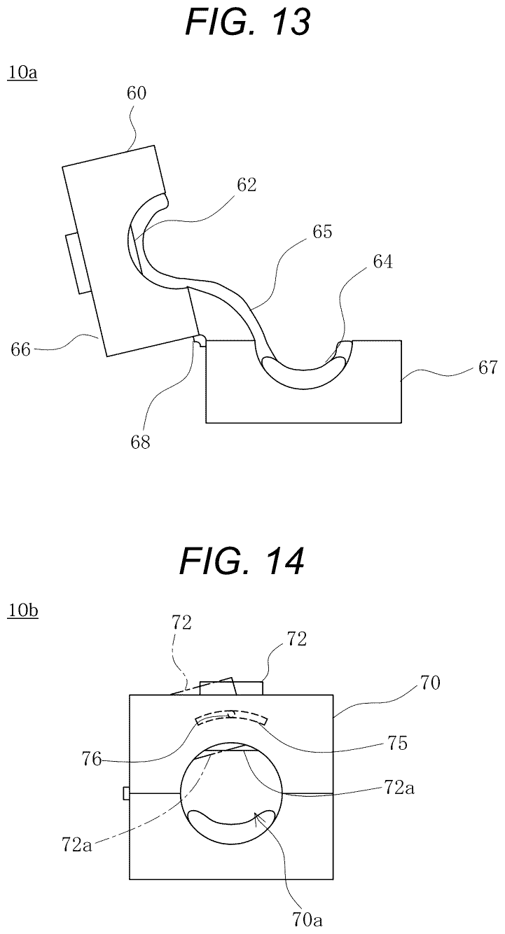

As illustrated in FIG. 13, in the base portion 60, a first base member 66 and a second base member 67 are connected via a hinge portion 68. The entire circumference pressing portion 65 is formed to be opened at a position in the circumferential direction. As a result, the first base member 66 and the second base member 67 can be opened even if the entire circumference pressing portion 65 is provided.

Next, an imaging apparatus 10b of a second modification will be described. As illustrated in FIG. 14, a slit 75 along the circumferential direction is formed inside a base portion 70 of the imaging apparatus 10b of the present modification. A probe body 72 has a support portion 76 to be engaged with the slit 75 and is supported on the base portion 70 by the support portion 76. Further an imaging surface 72a of the probe body 72 is exposed to an internal space 70a of the base portion 70. The support portion 76 can move along the slit 75 in the circumferential direction. Accordingly, as indicated by an alternate long and short dash line in the drawing, the imaging surface 72a of the probe body 72 moves in the circumferential direction with respect to the internal space 70a. As a result, after the arm H is inserted into the base portion 70, the imaging surface 72a can be moved in the circumferential direction with respect to the arm H. The probe body 72 can be moved in this manner, and thus, in a case where the cross-sectional image of the arm is displayed on the display unit 50, the probe body 72 can be moved in the circumferential direction to search for the blood vessel in a case where the blood vessel is not depicted.

In this example, the probe body 72 is supported so as to be movable in the circumferential direction with respect to the base portion 70, but may be supported along the length direction of the base portion 70. In addition, the probe body 72 may be supported by a free joint with respect to the base portion 70. In this case, an angle and a position of the probe body 72 can be changed along a plurality of axial directions with respect to the base portion 70.

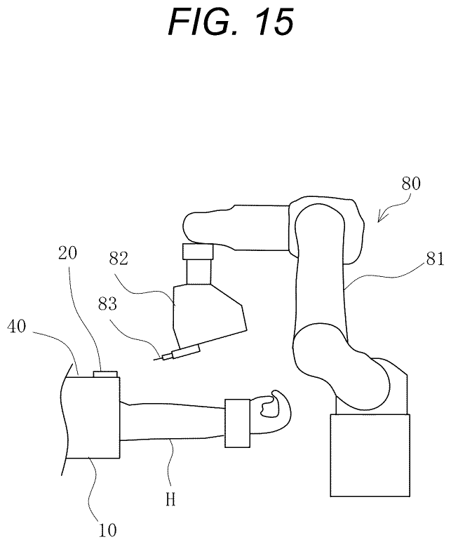

The imaging apparatus 10, 10a, or 10b described above can also be applied to an automatic puncture apparatus 80. As illustrated in FIG. 15, the automatic puncture apparatus 80 includes a robot arm 81 capable of three-dimensionally moving a distal end 82 to which a needle 83 is attached, and the imaging apparatus 10, 10a, or 10b. The robot arm 81 can perform puncture with the needle 83 from a particular position at an appropriate angle (e.g., 30 degrees with respect to the blood vessel) by control based on a sensor (not illustrated).

If the arm H is inserted into the base portion 40, 60, or 70 and held by the pressing portion 45, a cross-sectional image is acquired by the probe body 20, and the controller 30 detects the position of the blood vessel, the direction of the blood vessel, and the puncture depth from the cross-sectional image. In addition, a position and an angle at which puncture is to be performed with the needle 83 are determined on the basis of these. The robot arm 81 punctures the arm H with the needle 83 according to the determined puncture position and angle. Also in the automatic puncture apparatus 80, the arm H can be held by the pressing portion 45 or 65 (64) and pressed against the imaging unit 22, and thus, a cross-sectional image for detecting the position of the blood vessel can be reliably acquired.

As described above, the imaging apparatus 10 includes the probe body 20 having the imaging unit 22 that comes into contact with the skin surface to acquire a cross-sectional image of a human body, and the base portion 40 having a tubular shape in which the probe body 20 is provided and having the internal space 40a, and in the probe body 20, the imaging surface 20a along which the imaging unit 22 is disposed is exposed to the internal space 40a, the base portion 40 has the pressing portion 45 to face the imaging surface 20a of the internal space 40a, and the pressing portion 45 operates to reduce the internal space 40a of the base portion 40 by at least the surface 45a approaching the imaging surface 20a. In the imaging apparatus 10 configured as described above, the human body inserted into the base portion 40 can be pressed against the imaging surface 20a by the pressing portion 45, so that a clear cross-sectional image can be acquired by the imaging unit 22 being reliably brought into close contact with the human body.

Further, the pressing portion 45 may operate such that its volume expands toward the inside of the base portion 40. This can easily adjust a pressure to be applied to the human body from the pressing portion 45.

Further, the base portion 60 may have the entire circumference pressing portion 65 extending over the entire circumference of the inner surface in part of the internal space 60a. This can more reliably hold the human body in the base portion 60.

In the base portion 40, the inner surface forming the internal space 40a may be divided in the circumferential direction by the first base member 42 and the second base member 43, and the internal space 40a may be opened by mutually opening the first base member 42 and the second base member 43. This can easily take the human body in and out from the internal space 40a of the base portion 40.

In addition, the probe body 72 may be supported such that the imaging surface 72a can move in the circumferential direction of the internal space 70a with respect to the base portion 70. This can search for the blood vessel by moving the imaging surface 72a in the circumferential direction in a state where the human body is inserted into the base portion 70.

Further, the controller 30 may detect the sharpness of the cross-sectional image acquired by the imaging unit 22 and operate the pressing portion 45 so as to further reduce the internal space of the base portion 40 in a case where the detected sharpness is lower than a predetermined threshold. As a result, in a case where the pressing force of the pressing portion 45 against the human body is small, by adjusting the pressing force, it is possible to acquire a clearer cross-sectional image. Further, the predetermined threshold does not have to be provided, and the sharpness may be determined by machine learning.

Furthermore, in a case where the controller 30 detects that the blood vessel is deformed from the cross-sectional image acquired by the imaging unit 22, the controller 30 may operate the pressing portion 45 so as to make the internal space 40a of the base portion 40 larger. As a result, in a case where the pressing force against the human body by the pressing portion 45 is large, by adjusting the pressing force, it is possible to perform puncture while improving a state of the blood flow.

In addition, the controller 30 may operate the pressing portion 45 so as to make the internal space 40a of the base portion 40 larger in the moving direction of the blood vessel or in the direction opposite to the moving direction of the blood vessel in a case where the controller 30 detects that the position of the blood vessel is moved from the cross-sectional image acquired by the imaging unit 22. This makes it possible to prevent change in the position of the blood vessel, and reliably perform puncture.

Further, the imaging apparatus 10 may include the opening/closing detection unit 38 that detects an opened/closed state of the first base member 42 and the second base member 43, and if the opening/closing detection unit 38 detects that a state of the first base member 42 and the second base member 43 changes from a mutually opened state to a closed state, the controller 30 may operate the pressing portion 45 so as to reduce the internal space 40a of the base portion 40 after a certain period of time. As a result, when the human body is placed on the base portion 40, the cross-sectional image can be automatically acquired, which can reduce workload of the operator.

Note that the present invention is not limited to the above-described embodiments, and various modifications can be made by those skilled in the art within the technical idea of the present invention. For example, the acquired cross-sectional image is displayed on the display unit 50 such as a monitor. The controller 30 may detect the position of the blood vessel to calculate a puncture position and control the monitor provided on the probe body 20 to display the puncture position.

Furthermore, in the above-described embodiments, each of the pressing portions 45, 65, and 64 is formed with a cuff that can be inflated and deflated, but any pressing mechanism may be used as long as the arm H can be pressed against the imaging surface 20a by reducing the internal space 40a.

What is claimed is:

1. An imaging apparatus comprising:
a base portion having a space in which a part of a human body is placed;
a probe body attached to the base portion and including an imaging unit that faces the space and is configured to acquire a cross-sectional image of the part of the human body when the part of the human body is placed in the space;
a first pressing portion attached to the base portion and by which the part of the human body can be pressed against the imaging unit; and
a controller configured to control a pressure applied to the part of the human body by the first pressing portion.

2. The imaging apparatus according to claim 1, wherein the controller controls the pressure based on a quality of the cross-sectional image acquired by the imaging unit.

3. The imaging apparatus according to claim 1, wherein the first pressing portion is inflatable.

4. The imaging apparatus according to claim 1, further comprising:
a second pressing portion attached to the base portion and by which the part of the human body can be pressed, wherein
the space has a tubular shape, and the second pressing portion surrounds the space.

5. The imaging apparatus according to claim 1, wherein
the space has a tubular shape, and
the base portion includes first and second base members connected along a longitudinal direction of the space and movable to open and close the space.

6. The imaging apparatus according to claim 5, further comprising:
a sensor by which a closed state of the space can be detected, wherein
the controller is configured to increase the pressure a certain period of time after the closed state is detected by the sensor.

7. The imaging apparatus according to claim 1, wherein
the space has a tubular shape, and
the base portion includes a slit formed along a circumferential direction of the space and with which the probe body can engage such that an orientation of the imaging unit can be changed.

8. The imaging apparatus according to claim 1, wherein the controller is configured to:
determine a sharpness of the cross-sectional image, and
when the determined sharpness is lower than a threshold, increase the pressure, and then control the imaging unit to acquire another cross-sectional image of the part of the human body.

9. The imaging apparatus according to claim 8, wherein the controller is configured to:
determine whether a blood vessel in the cross-sectional image is deformed, and
upon determining that the blood vessel is deformed, decrease the pressure, and then control the imaging unit to acquire another cross-sectional image of the part of the human body.

10. The imaging apparatus according to claim 1, wherein the controller is configured to:
determine whether a position of a blood vessel in the cross-sectional image has changed from a previously acquired cross-section image of the part of the human body, and
upon determining that the position has changed, increase or decrease the pressure depending on a direction of the change in the position of the blood vessel.

11. An imaging apparatus comprising:
a base portion that includes a tabular space in which a part of a human body is placed;
an imaging unit disposed on an inner surface of the base portion that surrounds the space, the imaging unit being configured to acquire a cross-sectional image of the part of the human body when the part of the human body is placed in the space;
a first inflatable portion disposed on the inner surface of the base portion and facing the imaging unit; and
a controller configured to:
when the cross-sectional image is acquired by the imaging unit, determine whether a quality of the cross-sectional image satisfies a predetermined condition, and
upon determining that the quality does not satisfy the predetermined condition, inflate or deflate the first inflatable portion and then control the imaging unit to acquire another cross-sectional image of the part of the human body.

12. The imaging apparatus according to claim 11, wherein
the controller is configured to determine a sharpness of the cross-sectional image, and
the controller determines that the quality does not satisfy the predetermined condition when the sharpness is lower than a threshold.

13. The imaging apparatus according to claim 12, wherein
the controller is configured to determine whether a blood vessel in the cross-sectional image is deformed, and
the controller determines that the quality does not satisfy the predetermined condition when the blood vessel is deformed.

14. The imaging apparatus according to claim 11, wherein
the controller is configured to determine whether a position of a blood vessel in the cross-sectional image is changed, and
the controller determines that the quality does not satisfy the predetermined condition when the blood vessel is changed.

15. The imaging apparatus according to claim 11, wherein the base portion includes a slit formed along the inner surface and along which the imaging unit can be moved.

16. An automatic puncture apparatus comprising:
a base portion having a space in which a part of a human body is placed;
a probe body attached to the base portion and including an imaging unit that faces the space and is configured to acquire a cross-sectional image of the part of the human body when the part of the human body is placed in the space;
a first pressing portion attached to the base portion and by which the part of the human body can be pressed against the imaging unit;
a controller configured to control a pressure applied to the part of the human body by the first pressing portion; and
a robot arm to which a needle is attached and configured to perform a puncture operation on the human body based on the cross-sectional image.

17. The automatic puncture apparatus according to claim 16, wherein the controller controls the pressure based on a quality of the cross-sectional image acquired by the imaging unit.

18. The automatic puncture apparatus according to claim 16, wherein the first pressing portion is inflatable.

19. The automatic puncture apparatus according to claim 16, further comprising:
a second pressing portion attached to the base portion and by which the part of the human body can be pressed, wherein
the space has a tubular shape, and the second pressing portion surrounds the space.

20. The automatic puncture apparatus according to claim 16, wherein
the space has a tubular shape, and
the base portion includes first and second base members connected along a longitudinal direction of the space and movable to open and close the space.
